(19) United States
(12) Patent Application Publication
(10) Pub. No.: US 2024/0100205 A1
(43) Pub. Date: Mar. 28, 2024

(54) SANITIZATION APPARATUS AND DOORKNOB

(71) Applicant: Beniko ARAI, Tokyo (JP)

(72) Inventor: Dai ARAI, Tokyo (JP)

(21) Appl. No.: 17/953,240

(22) Filed: Sep. 26, 2022

(51) Int. Cl. A61L 2/04; A61L 2/26; C08J 5/18; E05B 1/00; F25B 21/02

(52) U.S. Cl. CPC A61L 2/04; A61L 2/26; C08J 5/18; E05B 1/0069; F25B 21/02; A61L 2202/14; C08J 2379/08

(57) ABSTRACT

An object surface is substantially cleaned by performing heating disinfection on a thin film at a high temperature in a state not contacting the object surface, and then covering and placing the thin film on the object surface. As another method, the object surface is cleaned by performing high-temperature heating on the object surface in a position retreated from its position of use, and then placing the object surface in the position of use again after cooling.

### CROSS-REFERENCE TO RELATED APPLICATIONS

The content of the following Japanese patent application is incorporated herein by reference:

NO. 2020-067893 filed in JP on Mar. 18, 2020 (Not Claimed)

### 1. TECHNICAL FIELD

The present invention relates to a technique of inactivating or reducing a pathogen or the like adhered to an object surface.

### 2. RELATED ART

Even in Japan, which is relatively in good hygienic conditions as compared to foreign countries, there is no end to infectious diseases due to a seasonal flu or a norovirus every year. In addition, during recent years, outbreaks of a new flu, a new coronavirus, and the like also became new threats to humans. Although their infection routes are diverse, contact infection can be mentioned as one of common infection routes. That is, a route of indirect contact infection resulting from a pathogen on a hand or the like of a carrier adhering to an object surface due to a touch to a doorknob, a handrail, or the like by the carrier, followed by a touch by a healthy individual.

A method of disinfection by wiping using a chemical such as ethanol or sodium hypochlorite can be primarily mentioned as a method conventionally applied to prevent such contact infection. However, disinfection with a drug has no effect against a pathogen that does not conform to a disinfection spectrum of the drug, and a certain period of time (several tens of seconds to several minutes) is required for the drug to disinfect the pathogen. In the first place, a method of frequent disinfection, for example, disinfection by wiping with a drug every time a user grips a doorknob, is not a realistic means.

As one conventional technique for this problem, products with surface treatment appealing antibacterial or antiviral have been released in the world. Such products often use silver ions, special polymers, or the like. However, many of such products show a performance of only slightly suppressing propagation of an adhered pathogen as compared to an untreated surface, or a performance of only slowly reducing the pathogen over several hours to several tens of hours. Accordingly, even if such products are employed for a common-use part such as a doorknob being frequently used, as a matter of practice, an effect of suppressing contact infection can hardly be expected.

As described above, among conventional techniques/products, a technique/product that rapidly inactivates or reduces, in about several seconds to several tens of seconds, a pathogen on an object surface having a possibility of frequent adherence of a pathogen such as a doorknob, does not exist.

### PRIOR ART DOCUMENT

### Non-Patent Document

Non-Patent Document 1: “Issues Concerning Survival of Viruses on Surfaces”, P. Vascickova, et. al, 2010

### SUMMARY OF INVENTION

### Problem to be Solved by the Invention

The problem to be solved is to inactivate or reduce a pathogen adhered to an object surface, and achieve a cleaned state of the object surface by the time of next use of the object.

### SUMMARY

To solve the above-described problem, the present invention proposes a method and an apparatus as follows.

An object surface desired to be used as a cleaned state is temporarily retreated from a position of use, and a heating treatment is performed in the retreat position. Heating conditions (temperatures, heating times, and the like) at this time are arbitrary in accordance with requirements such as types of pathogens desired to be inactivated/reduced and levels of reduction.

The meaning of retreating the object surface also includes a configuration of covering the object surface with a cover or the like during heating.

Regarding the heating of the object surface, generally, the higher the heating temperature, the shorter the inactivation time of a pathogen is. Thus, a higher heating temperature is desired. On the other hand, in most cases, use is not possible if the temperature at the time of use is such high temperature. For example, although most pathogens are killed or inactivated if the doorknob is heated with 200° C. for 10 seconds, a person cannot touch the doorknob until it returns to a room temperature.

Thus, the present invention is focused on keeping a heat capacity of an object surface subjected to heating disinfection to the minimum necessary. The heat capacity that is minimum necessary means, heating a thin material such as a film or a thin plate. This is a means of heating only this thin material, and once a desired inactivation/reduction is achieved, placing this thin material on a base material or skeleton having a required rigidity.

According to this method, since a thin material to be disinfected only has a heat capacity that is minimum necessary, there is no risk of a burn injury even if a person's hand touches it immediately after heating to a high temperature. In addition, also when cooling is performed after the high-temperature heating, since an accumulated heat quantity is little, rapid heat dissipation (cooling) is realized as in the case of the heating. Furthermore, since a thin material has a wide area contacting the external air, it rapidly returns to a room temperature only with exposure to the air of a room temperature after the heating. Accordingly, by selecting a material having an appropriate specific heat and thickness, and by setting a heating temperature and a cooling time until a person touches the heated surface, there is no risk of a person suffering from a burn injury after the high-temperature heating.

In addition, this is applicable not only to disinfection of an object surface to be directly touched by a person, but also to an object that requires cleaning of its surface such as a cutting board. Since rapid cooling can be realized after heating to a high temperature also in this case, it is also applicable to foodstuff that is vulnerable to heat such as sliced raw fish. In this manner, since frequent disinfection can be performed on each foodstuff without the use of a chemical such as a detergent, and time can also be saved, food poisoning through a cutting board can be expected to be prevented.

Furthermore, heating only a thin material means that energy put into the heating can be extremely little. For example, with a heat quantity that raises water of 200 μm thickness by 25° C., an aluminum foil of 10 μm thickness can be raised by as high as 180° C. If a room temperature is set to 23° C., by raising the temperature by 180° C. and reaching 203° C., pathogens can be almost detoxified in about 10 seconds.

Describing the previous paragraph in another way, when an aluminum foil of 10 μm thickness that is heated up to 200° C. in a room temperature of 23° C. is removed from a heating body and a person touches it with his/her hands and fingers immediately after that, there will be no burn injury. This is because, since the aluminum foil promptly starts heat dissipation in the atmosphere of 23° C. immediately after being removed from the heating body and even if the heat is uniformly applied to the skin of hands and fingers of 200 μm, a temperature thereof only rises about 20° C., it will only feel like holding a teacup with tepid water.

As described above, at the time of performing heating disinfection on a surface of a structural body, it is extremely effective means in terms of disinfection efficiency and also in terms of energy saving to separate the surface as thin as possible and performing heating disinfection only on the separated surface with a high temperature.

The heating temperature and time will now be described. When qualitatively considered, there is no doubt that the longer hours the heating is performed with the higher temperature, inactivation/reduction of a pathogen can be expected. However, in the actual product design, setting and designing should be done in accordance with a requirement specification of the apparatus in consideration of efficiency and economics. There is one rough standard that heating should be performed to a boiling point of water or higher if sterilization is intended. Bacteria are microorganisms unlike viruses, and moisture is stored within a cell wall. Accordingly, if a bacterium is heated to the boiling point or higher, at the instant the water is vaporized, swelling occurs and the cell wall is destroyed due to internal pressure, and the bacterium can no longer survive. Therefore, if heating is performed to the boiling point of water or higher, i.e., 100° C. or higher in the atmosphere, and adhered bacteria can be brought to a state of 100° C. or higher, the bacteria can be instantly killed.

On the other hand, this does not necessarily apply to viruses. Since viruses and phages are merely chemical substances and not organisms, their molecular structures may be maintained for a while even in an environment of 100° C. or higher. Therefore, the heating temperature may be determined in view of a heatproof temperature of a thin film such as an aluminum foil to be used, a temperature of a heating body, economics, and safety. In an experiment beforehand, when a ceramic heater for soldering iron, which is easily available and inexpensive as a heating body, is connected to a 12V power source and heated, the temperature is raised up to 180° C. in 10 and several seconds. Thus, from viewpoints of achievement of an inexpensive apparatus configuration and component procurement, heating at 180° C. for about 10 seconds will be one rough standard of heating conditions.

In addition, viruses include those having coatings called envelopes and those not having envelopes. For example, flu viruses and coronaviruses are types having envelopes, and noroviruses are types not having envelopes. Although intuitively those having coatings seem to have more external resistance, actually they do not. This is because the envelope types can be inactivated by only rupturing its envelopes. On the other hand, the non-envelope types cannot be inactivated unless its structure itself is destroyed. Accordingly, regarding the envelope types, it can be expected that moisture inside an envelope will be vaporized and the envelope will be destroyed from the inside by heating to the boiling point of water or higher, as in the case of heating disinfection of bacteria.

In other words, as a rough standard of heating disinfection against bacteria and enveloped viruses, a heating temperature at the boiling point of water or higher is effective, and for non-envelope viruses, a heating temperature at 180° C. or higher is efficient from a viewpoint of availability of apparatus components. These are rough standards at the time of practical design of an apparatus.

### Effect of the Invention

According to the present invention, a surface of an object that requires cleaning can be rapidly and automatically disinfected/attenuated in an energy-saving way. In this manner, it is possible to satisfy social needs requiring cleaning for each opportunity of contamination, such as cleaning of doorknobs, handrails, toilet seats, surfaces of cutting boards and the like where their object surfaces are frequently contaminated.

### BRIEF DESCRIPTION OF THE DRAWINGS

FIG. 1A is, among schematic configurations of a gripping structure of a doorknob or the like, a perspective view of the gripping structure which is one example of an embodiment of the present invention.

FIG. 1B is a perspective view of a cross section of a gripping portion, among the schematic configurations of the gripping structure of the doorknob or the like.

FIG. 1C is a perspective view of a state in which a cover of a driving part is removed, among the schematic configurations of the gripping structure of the doorknob or the like.

FIG. 1D is a cross-section schematic diagram of the gripping portion, among the schematic configurations of the gripping structure of the doorknob or the like.

FIG. 2 is a schematic configuration of a push/pull structure of the doorknob or the like, which is one example of an embodiment of the present invention.

FIG. 3A is one example of an embodiment in which the form of FIG. 2 is divided into half, and is an example of use in a simple push part such as a door or a push button.

FIG. 3B is one example of an embodiment in which the form of FIG. 2 is divided into half, and is an example of use in a cutting board or a temporary storage space of a medical instrument such as a surgical tool by using it horizontally.

FIG. 3C is one example of an embodiment in which the form of FIG. 2 is divided into half, and it illustrates a configuration using a Peltier element.

FIG. 4 is a configuration example of application to a handrail of an escalator.

FIG. 5 is one example of a configuration in which an object surface is not retreated but is temporarily covered with a lid structure when performing heating.

FIG. 6 is one example of a conceptual diagram of a basic configuration of an embodiment of the present invention, which illustrates an example using a film.

FIG. 7 is one example of a conceptual diagram of a basic configuration of an embodiment of the present invention, which illustrates an example using a thin plate.

FIG. 8 is a schematic diagram representing a configuration of an eighth embodiment.

FIG. 9 is a schematic diagram representing a configuration of a ninth embodiment.

### DESCRIPTION OF EXEMPLARY EMBODIMENTS

The core intent of the present invention is to perform heating disinfection of only a minimum thickness part of a surface of a functional structural object (handrails, cutting boards, toilet seats, and the like) for which cleaning is desired. However, if a part for which cleaning is desired is made too thin or too small, problems of rigidity and durability may occur. On the contrary, if the part is made thick to a certain level, there are disadvantages that frequent use cannot be withstand because long heating time and cooling time are required, and great energy is required.

Accordingly, the thickness and the structure of an apparatus should satisfy heating conditions based on design requirements, that is, within how many seconds pathogens are reduced and how much the pathogens are reduced upon returning to a reuse state. Since there are countless numbers of such design achieving means, it cannot be uniformly determined. The following embodiments show only one example using the present invention among those configurations that can withstand practical design.

### First Embodiment

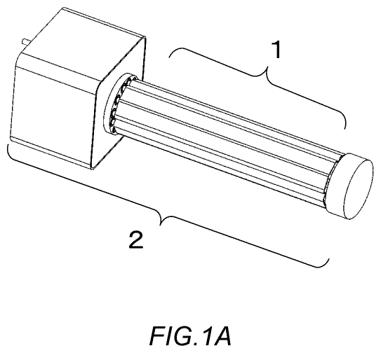

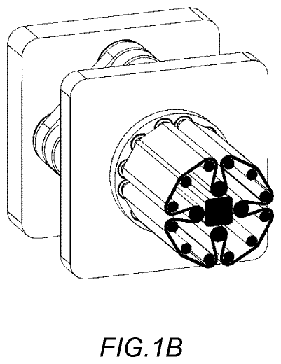

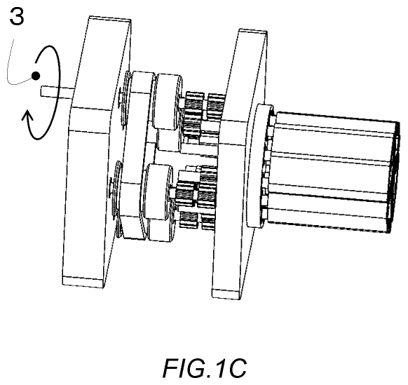

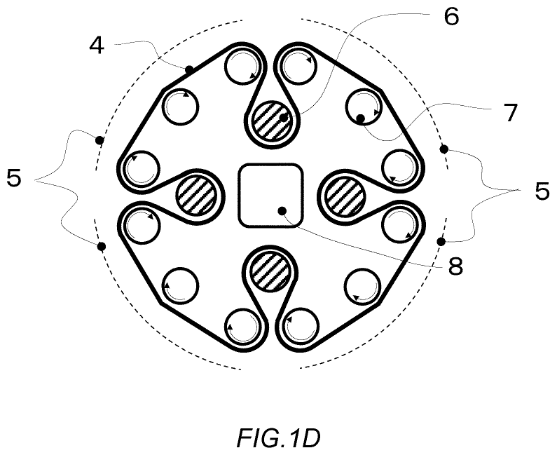

FIG. 1A to FIG. 1D are one embodiment of the present invention, and these illustrate embodiments in a gripping structural body to be gripped, pushed, or pulled such as a doorknob, a handrail, a hanging strap, a key of a door, or the like. As shown in FIG. 1B and FIG. 1D, a gripping portion 1 is formed of a supporting structural body 8, a rotary driving support 7, a heating body 6, and a thin film 4. One or a plurality of rotary driving supports 7 is driven to rotate by a rotary actuator 3 shown in FIG. 1C to cause the thin film 4 to run along the structural body.

The thin film 4 is disinfected/attenuated when contacting and being heated by the heating body 6, which is placed in a position retreated from a surface at the time of use 5 (i.e., in this case, a surface of the gripping portion where a skin contacts at the time of gripping by a user). Then, the disinfected/attenuated thin film 4 is caused to run to the surface at the time of use 5 again.

The heating body 6 at this time may be an electrically-heated wire such as a nichrome wire or a ceramic heater, or flames may be indirectly applied like a burner type iron. Alternatively, indirect heating may be performed by letting a fluid of a high temperature flows in a pipe.

The thin film 4 maintains its place with rigidity by being supported by the rotary driving support 7. If the gripping structural body is a one end supported structure, its rigidity is retained by the supporting structural body 8 and the rotary driving support 7 placed inside the gripping portion. Although the same applies to a case where the gripping structural body is supported at both ends, since the supporting structural body 8 can be formed by pulling from both sides, it is possible to form it alternatively with a lightweight member such as a wire.

A gripping structural body 2 automatically detects its use. A detection method may be a vibrometer attached to the main body, a strain gauge that detects occurrence of a strain due to gripping, or a non-contact sensor of an optical detection type. Alternatively, a switch that a user can actively operates or a microphone that reacts to a voice command may also be provided.

The gripping structural body 2 detecting its use also similarly detects a state in which use is finished such as a user letting go his/her hand, and immediately after the completion of use or after a certain period of time, starts running of the thin film 4. The heating body 6 is also heated at this time, and by heating the thin film 4 contacting and passing the heating body 6, disinfection/attenuation is sequentially performed.

Once the entire surface of gripping use becomes a disinfected/attenuated state, the thin film 4 stops running, and a reusable state is achieved.

At this time, for some or all of the reusable state, the used state by a user, the state in which the use by the user is completed, the state in which the gripping structural body is performing a disinfection process, a function of lighting a lamp or issuing a voice to indicate the state is also equipped.

If a contact length of the heating body 6 and the thin film 4 is longer than a length of the surface at the time of use 5 of the thin film 4, a heating treatment on the thin film 4 can be concurrently performed also when a user is using the gripping structural body 2. In this manner, the thin film 4 can be caused to quickly run after the completion of use of the gripping structural body 2 to be prepared for the next use. That is, since the time during use and the time of heating disinfection can be concurrent, a tact time can be shortened.

If a contact length of the heating body 6 and the thin film 4 is not longer than a length of the surface at the time of use 5 of the thin film 4, it is also certainly possible to concurrently perform a heating treatment on the thin film 4 while a user is using the gripping structural body 2. However, a part in the thin film 4 other than a part for which the heating treatment is concurrently performed should be subjected to heating disinfection by causing the thin film 4 to sequentially run and contact the heating body 6 after the completion of use by the user.

At this time, the thin film 4 may be rotated only in a same direction, or may be reversed. In addition, although a starting point and an ending point of the thin film 4 is joined to have a loop shape in FIG. 1D, the thin film 4 may be opened without a joint and used by reversing it after winding in one direction or may be used from one direction again after winding it all back. In other words, for example, this means a method of use in which after playing one side of a cassette tape from one direction, the tape may be played in a reverse direction by an auto-reverse function, or the same one side may be played again by winding it all back after playing one side.

It should be noted that the heating time and temperature are being measured, and there is a temperature control so that a desired inactivation/reduction of a pathogen is performed and overheating of an apparatus including burn injuries and breakdowns is prevented.

The thin film 4 may be a resin film besides an aluminum foil, or may be a film in which metal such as an aluminum foil is vapor-deposited on a resin film. A polyimide film can be mentioned as one example of the resin film. For example, a polyimide film of DuPont that is known in the trade name of Kapton film (registered trademark) has a heatproof temperature of approximately 400° C., and it can sufficiently withstand a heating temperature up to about 200° C. which is assumed in the present invention. In addition, since there are also many types of film thickness, a thickness that is suitable for a product specification thereof can be selected from viewpoints of a residual heat quantity/temperature at the time of use and durability. Besides these, the thin film 4 may be any film as long as it is a material that can withstand an apparatus heating specification temperature.

As described above, cleaning of the gripping portion 1 is performed by using the present invention. Along with this, a moisture adhered to a surface can also be evaporated by heating. As often seen in a doorknob of a restroom or the like, when a previous user touches the doorknob with a wet hand, water droplets may be left on the doorknob. Such discomfort and uncleanliness can also be secondarily solved by the present invention.

### Second Embodiment

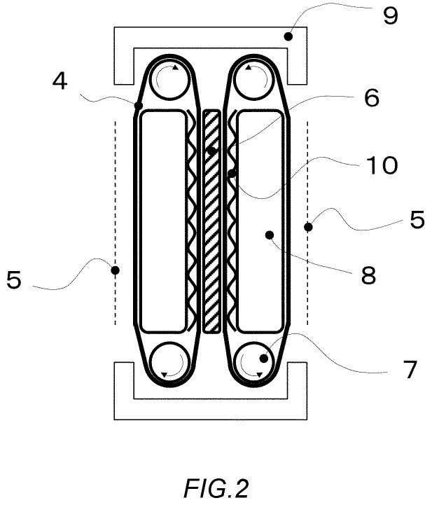

FIG. 2 shows another embodiment. This embodiment has a configuration in which a front side and a rear side of a plane can be disinfected. It is applicable to a structural body that is pushed from both surfaces or grabbed by both surfaces such as a pushing plate of a door.

In this configuration, the heating body 6 is placed inside, at the center. As in the case of the first embodiment, once a use of the surface at the time of use 5 is detected, the thin film 4 is caused to run and is positioned in the heating body 6 after the completion of use. At this time, as in the case of the first embodiment, if a contact length of the thin film 4 and the heating body 6 is sufficiently long, heating disinfection can be performed in advance when the thin film 4 is not running such as at the time of use or at the time of waiting of the apparatus, and thus time can be saved.

### Third Embodiment

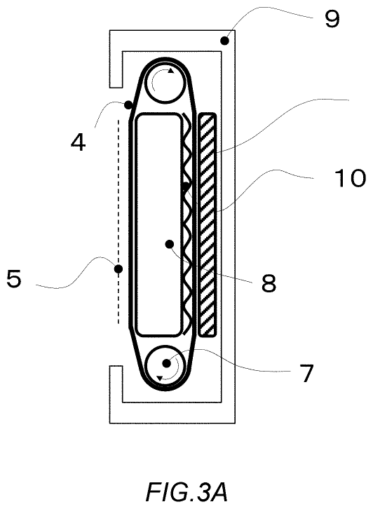

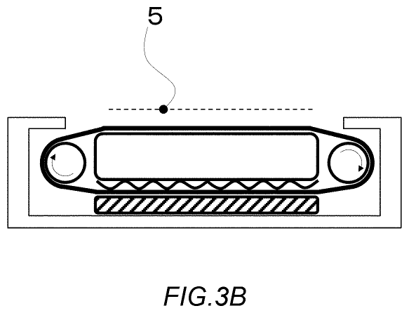

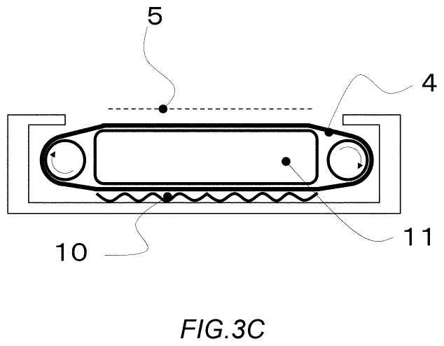

FIG. 3A shows an embodiment 3A as a variation of the second embodiment. In this embodiment, only one side of the second embodiment is used. This configuration may be used when cleaning of both surfaces is not required. However, since the heating body 6 generally generates heat on both sides, a heat quantity on the opposite surface may be wasted or a mechanism of externally releasing the heat of a housing or the like that is raised by the heat generation may be required.

Besides the use in a structure as a member to be contacted for simply pushing such as a pushing plate or a push button of a door, this embodiment can also be used by being horizontally placed as in FIG. 3B. A cutting board for cooking, a stand for temporarily putting on a medical instrument such as a surgical instrument, and the like for which cleanliness is required can be mentioned as application examples of the case of horizontal placing.

FIG. 3C shows an embodiment 3C as a further variation. In this embodiment, the thin film 4 is pressed to a Peltier element 11 by a pressing elastic body 10. The Peltier element 11 is placed and used while setting the lower side in the figure as a heating side and the upper side in the figure as a cooling side. In this manner, even in a condition that the thin film 4 after heating is not cooled to a room temperature, it can be forcibly cooled on the upper surface of the Peltier element 11. In addition, also when applying to a structural object that is vulnerable to afterheat such as a cutting board, this structure is beneficial because a surface at the time of use is retained at a room temperature or lower.

### Fourth Embodiment

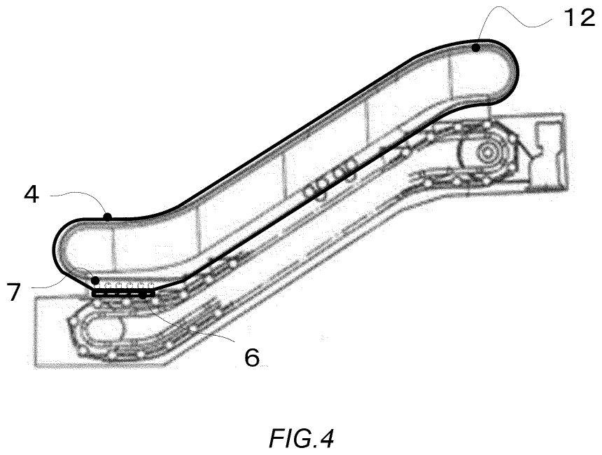

FIG. 4 shows a fourth embodiment. This is an embodiment in which the present invention is applied to a handrail (moving handrail) 12 of an escalator. The handrail 12 is a structural body to be frequently touched with hands of an unspecified large number of people. Furthermore, an extremely speedy disinfection treatment is required.

When heating the handrail 12 itself such as that used in an existing escalator, it is difficult to perform heating in a short period of time due to a great heat capacity. Even if heating can be performed in a short period of time by applying a high temperature, a cooling time until returning to a room temperature to be touched by a person is insufficient. Thus, in the fourth embodiment, the thin film 4 is covered on the handrail 12 by using a system of the present invention.

At a time when a user puts a hand on the handrail 12, the handrail 12 and the thin film 4 are in contact and are moving integrally. However, in a returning path where the user is not holding the handrail 12, the handrail 12 and the thin film 4 are separated. Furthermore, only the thin film 4 is disinfected by being heated for a short period of time at a high temperature by the heating body 6. After the heating disinfection, the thin film 4 is brought into contact and integrated with the handrail 12 again. A heat quantity accumulated in the thin film 4 in accordance with the heating treatment moves to the handrail 12 or disperses in the air, and is promptly turned into a room temperature.

In this manner, the user of the escalator can always touch the handrail 12 that has been disinfected.

### Fifth Embodiment

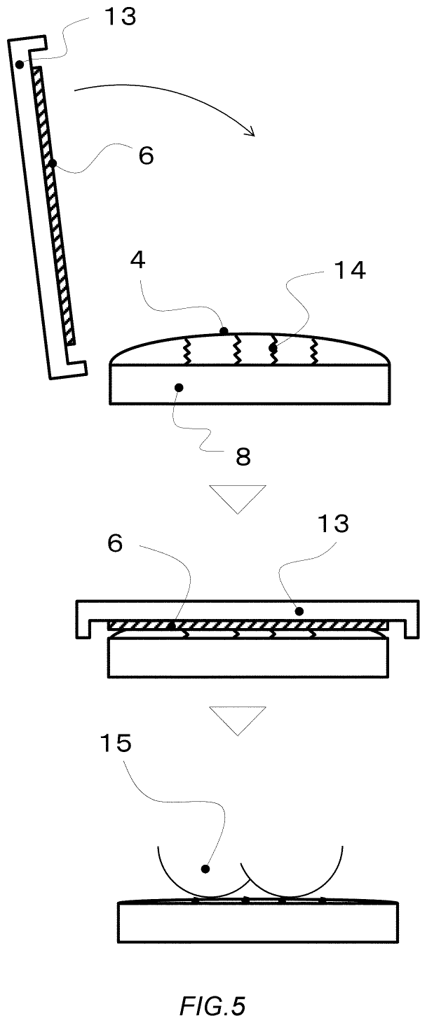

FIG. 5 shows a fifth embodiment. Although the embodiments described above caused the thin film 4 to run and performed a heating treatment in a position other than a surface at the time of use, the thin film 4 is not caused to run in this embodiment.

The thin film 4 is joined with the supporting structural body 8, and as in FIG. 5, it can be floated from the supporting structural body 8 as a structure having a small heat transfer quantity by using an elastic body 14. The elastic body 14 herein may be a fine metal spring or a resin spring having a low heat transfer coefficient, or it may be inflated with any gas like a balloon. What is important is that, in the following heating treatment process, a quantity of heat used for the heating of the thin film 4 transferring to the supporting structural body 8 through the elastic body 14 is minimized.

At the time of the heating treatment, the heating body 6 placed on the inner side of a lid structure 13 contacts the thin film 4 and heats the thin film 4. At this time, the thin film 4 is floated from the supporting structural body 8 by the method described above such that heat transfer is minimized.

After a predetermined heating treatment is completed, the thin film 4 has been disinfected when the lid structure 13 is removed, and a user can use the supporting structural body 8 covered with the disinfected thin film 4.

An application to a toilet seat in a restroom can be mentioned as one example of this embodiment. In this figure, the application is expressed as an example simulating a toilet seat and a toilet seat cover. In this case, a contact object 15 is the buttocks of a person, and the supporting structural body 8 is a toilet seat. Besides this, it is similarly applicable to a cutting board, a storage space of a medical instrument, and the like for which cleanliness is required.

### Sixth Embodiment

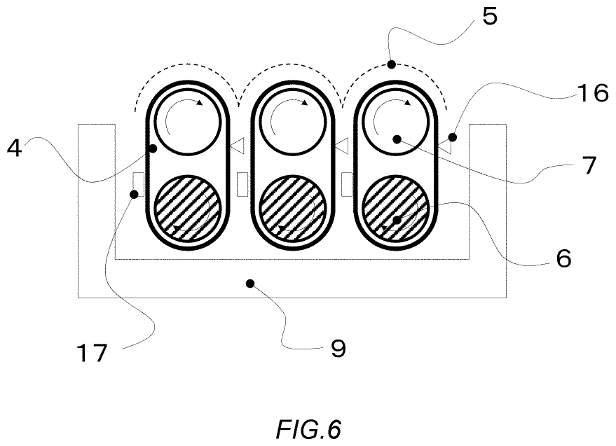

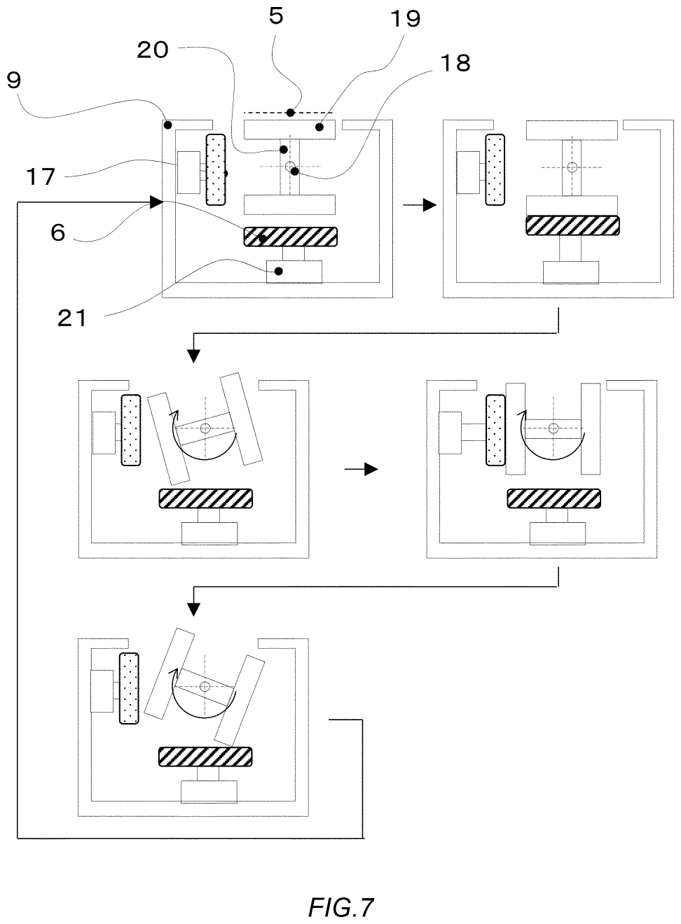

FIG. 6 and FIG. 7 show examples of very basic configurations of an embodiment of the present invention. FIG. 6 shows an achieving means using the thin film 4. In this figure, the heating body 6 is placed in a position hidden from the surface at the time of use 5. Therefore, a user will not accidentally touch the thin film 4 at the time of heating. The heated and disinfected thin film 4 is caused to run to the surface at the time of use 5 by the rotary driving support 7 to be prepared for a use state. Whether the apparatus is being used may be confirmed with the use detecting sensor described above. Even if the apparatus is not used at all, a heating process may be performed at every arbitrary time to eliminate a pathogen adhered by airborne droplets or the like.

In addition, there may be a case in which contamination at the time of use involves fine powders exceeding several hundreds of μm or a case in which a microorganism forms a biofilm on a film. In such case, it is assumed that a heat of the heating body 6 is not sufficiently transferred, and the temperature does not rise to a predetermined temperature. To remove such relatively large particle contaminants in advance, a surface contaminant sweeping removal apparatus 16 using a scraper or a brush may be placed before sending the thin film 4 to the heating body 6.

In addition, if the thin film 4 does not promptly return to a room temperature by natural cooling, a cooling body 17 may be provided in advance and cooling may be performed after heating of the thin film 4 is finished. Alternatively, the thin film 4 may be promptly cooled to a room temperature by cooling the rotary driving support 7 supporting the surface at the time of use 5. In addition, the thin film of the present invention does not necessarily refer only to a uniform thin film product of resin, but it may be a metal foil such as an aluminum foil or a metal may be vapor-deposited on a resin thin film. Furthermore, it may be a sturdy paper represented by Japanese papers. Alternatively, it may be a thin fabric using a natural material or an artificial material. There is no need for weaving as a fabric, and stringlike objects may be merely thinly placed in parallel or in a random manner. That is, things that are thinly processed to reduce a heat capacity are generally called thin films in the present invention.

### Seventh Embodiment

FIG. 7 shows an embodiment of the present invention when the thin film 4 is not used. A contact surface body 19 having a minimum heat capacity is supported by a contact surface supporting structural body 20 having a small heat transfer quantity. After a user uses the surface at the time of use 5, the contact surface body 19 retreats from the surface at the time of use 5 by a rotational bearing 18, and moves to the heating body 6. A retreat method of the contact surface body 19 at this time may be linear driving regardless of the rotational bearing 18. The contact surface body 19 moved to the heating body 6 contacts the heating body 6 and receives a heating treatment. After receiving the heating treatment, if a temperature drop of the contact surface body 19 is insufficient, it moves to the position of the cooling body 17 and receives heat dissipation by the cooling body 17. Then, the contact surface body 19 is sent to the surface at the time of use 5 again to be prepared for the next use.

FIG. 7 illustrates a case of having two contact surface bodies 19, but a plurality (such as three, four, or five) of contact surface bodies 19, the heating body 6, and the cooling body 17 may be prepared. Although the apparatus becomes larger, a tact time can be shortened.

### Eighth Embodiment

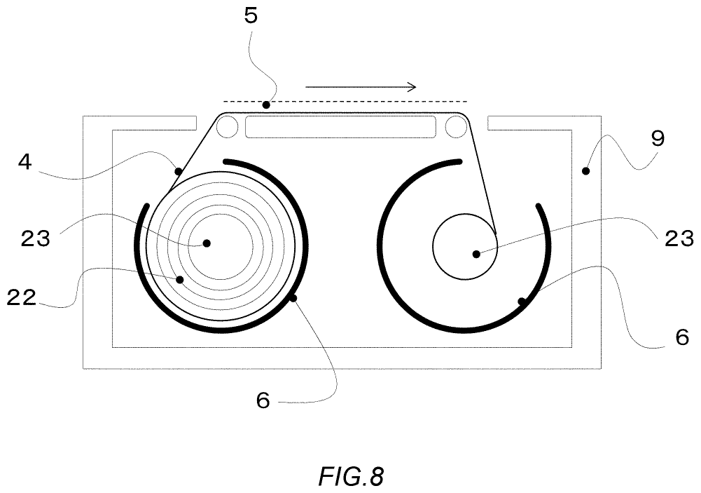

An eighth embodiment will be described by using FIG. 8. The eighth embodiment resembles a driving method of a cassette tape. In FIG. 8, the thin film 4 is in a state that is wound to a thin film reel 22 on the left side in advance. In this thin film reel 22, the entire reel can be heated with the heating body 6. Therefore, the thin film 4 is disinfected/attenuated by heating the entire reel with the heating body 6 during times when the apparatus is not being used such as at night. Then, at times of use of the apparatus, the thin film 4 is placed on the surface at the time of use 5 by being sequentially sent out from the thin film reel 22. At this time, the thin film 4 is wound by a rotary driving body 23. Meanwhile, the thin film 4 may be rotated only in a same direction, or may be reversed. In addition, although a starting point and an ending point of the thin film 4 is joined to have a loop shape as shown in the figure, the thin film 4 may be opened without a joint and used by reversing it after winding in one direction or may be used from one direction again after winding it all back. In other words, for example, this means a method of use in which after playing one side of a cassette tape from one direction, the tape may be played in a reverse direction by an auto-reverse function, or the same one side may be played again by winding it all back after playing one side.

In this manner, in the case of a reel winding system as in a cassette tape, a heating treatment during use becomes unnecessary by performing predetermined heating disinfection on the entire reel in advance, and a frequency of use can be further enhanced by being able to increase a running speed of the thin film.

### Ninth Embodiment

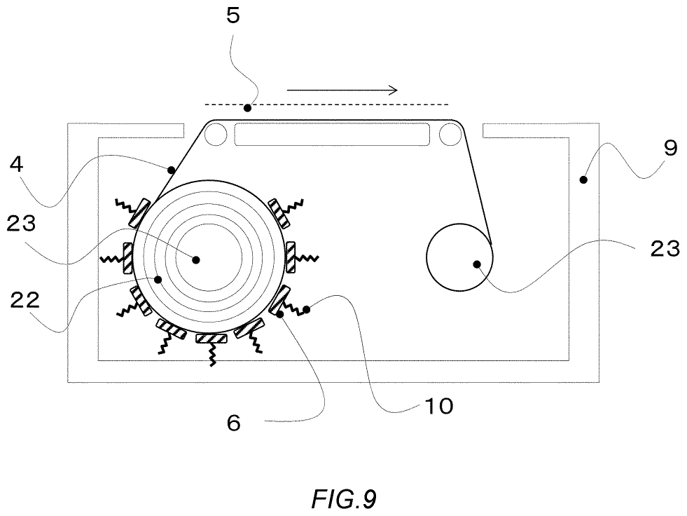

FIG. 9 shows a ninth embodiment. The ninth embodiment is a variation of the eighth embodiment. In FIG. 9, the heating body 6 is attached firmly to the thin film 4 in the outermost circumference of the thin film reel 22 by the pressing elastic body 10. The heating body 6 generates heat at the timing of performing heating disinfection on the thin film 4 in the outermost circumference. Since the heating body 6 is pressed to the outermost circumference of the thin film reel 22 by the pressing elastic body 10, the structure is such that heating disinfection is always performed on the thin film 4 in the outermost circumference even if a diameter of the thin film reel 22 decreases in accordance with the sending of the thin film 4.

It should be noted that although not illustrated, the ninth embodiment also has a cassette tape system as in the case of the eighth embodiment, and inversion driving may be performed after winding from the thin film reel 22 on the left side to the thin film reel on the right side, or normal driving may be performed after winding it all back to the thin film reel 22 on the left side.

### INDUSTRIAL APPLICABILITY

The present invention can be widely used for surfaces of structural bodies that always require cleanliness. As an example of general usage, it is used as a cleaning apparatus for a product to which an unspecified large number of people repeat contact in a short period of time such as a knob or a gripping portion of a door, a handrail, or a hanging strap. In addition, it can also be used for cleaning of a surface of a structural body not to be gripped such as a toilet seat in a restroom.

Regarding usage other than for people, there is usage for a cutting board or the like where frequent cleaning is required. Since attachment of a chemical such as a detergent is desired to be as little as possible, the method of the present invention is effective.

The present invention is also beneficial as usage for a medical instrument. In a general ward, it can be used for a handle of a door, a handrail of a bed, and the like in a hospital room to prevent hospital-acquired infections. It can also be used for a part where a hand is put on in a restroom. In addition, it can also be used as a placing stand when there is a desire to temporarily place a medical instrument during a physical examination or a surgery. Furthermore, it can also be used for a structural body that needs be touched or gripped while wearing gloves, which require cleanliness, during a surgery. The structural body is, for example, a grip portion or the like of a shadowless lamp in a surgery. The present invention may also be embodied by aspects according to the following items.

Item 1

A means of performing inactivation or reduction of a pathogen on a surface of a structural body, which is a method of seeking to inactivate or reduce the pathogen on the surface of the structural body by heating a covering member in a position not exposed to the surface of the structural body, and placing the covering member on a base material forming a skeleton of the structural body after a predetermined heating treatment has been completed.

Item 2

The means of performing inactivation or reduction of the pathogen on the surface of the structural body according to item 1, which is a method of seeking to inactivate or reduce the pathogen adhered to a surface of the covering member by heating the covering member to 100° C. or higher.

Item 3

The means of performing inactivation or reduction of the pathogen on the surface of the structural body according to item 1, which is a method of seeking to inactivate or reduce the pathogen adhered to a surface of the covering member by heating the covering member to 180° C. or higher.

Item 4

An apparatus using the means of performing inactivation or reduction of the pathogen on the surface of the structural body according to item 1, which is an apparatus seeking to inactivate or reduce the pathogen on the surface of the structural body characterized in that a thickness of the covering member is 30 micrometers or less.

Item 5

An apparatus using the means of performing inactivation or reduction of the pathogen on the surface of the structural body according to item 1, which is an apparatus seeking to inactivate or reduce the pathogen on the surface of the structural body characterized in that a ceramic heater is used for a heating body to heat the covering member.

Item 6

The means of performing inactivation or reduction of the pathogen on the surface of the structural body according to item 1, which is an apparatus seeking to inactivate or reduce the pathogen on the surface of the structural body characterized in that a reuse preparation time of the apparatus is shortened by performing heating disinfection/attenuation on the covering member by a length of one-time use or longer in advance, and placing the disinfected/attenuated covering member of a length for only one-time use in a position of use.

Item 7

A means of performing inactivation or reduction of a pathogen on a surface of a structural body, which is a method of seeking to inactivate or reduce the pathogen on the surface of the structural body by heating the surface of the structural body in a position not exposed to a surface in a use state of an apparatus, and moving the surface of the structural body for placement in a surface position in the use state of the apparatus after a predetermined heating treatment has been completed.

### EXPLANATION OF REFERENCES

1 gripping portion
2 gripping structural body
3 rotary actuator
4 thin film
5 surface at the time of use
6 heating body
7 rotary driving support
8 supporting structural body
9 support housing
10 pressing elastic body
11 Peltier element
12 handrail
13 lid structure
14 elastic body
15 contact object
16 surface contaminant sweeping removal apparatus
17 cooling body
18 rotational bearing
19 contact surface body
20 contact surface supporting structural body
21 linear motion actuator
22 thin film reel
23 rotary driving body

What is claimed is:

1. A sanitization apparatus comprising:
a heating part;
a part to be heated for heating by the heating part; and
a driving part for switching a heating position where the part to be heated is heated by the heating part, and a position on a surface of a structural body where the part to be heated is exposed.

2. The sanitization apparatus according to claim 1, wherein
the heating position is a position where the part to be heated is not exposed to the surface of the structural body.

3. The sanitization apparatus according to claim 1, wherein
the part to be heated comprises a thin film.

4. The sanitization apparatus according to claim 3, wherein
the thin film is a resin film.

5. The sanitization apparatus according to claim 1, wherein
the part to be heated is a polyimide film.

6. The sanitization apparatus according to claim 1, wherein
a thickness of the part to be heated is 30 μm or less.

7. The sanitization apparatus according to claim 1, wherein
the heating part is for heating the part to be heated to 100° C. or higher.

8. The sanitization apparatus according to claim 1, wherein
the heating part is for heating the part to be heated to 180° C. or higher.

9. The sanitization apparatus according to claim 1, comprising
a cooling part for cooling the part to be heated.

10. The sanitization apparatus according to claim 1, wherein
the heating part comprises a Peltier element for heating the part to be heated.

11. The sanitization apparatus according to claim 10, wherein
the Peltier element comprises a high-temperature part and a low-temperature part, and
the high-temperature part is for functioning as the heating part and the low-temperature part is for functioning as a cooling part for cooling the part to be heated.

12. The sanitization apparatus according to claim 1, comprising
an elastic part for causing the part to be heated to contact the heating part.

13. The sanitization apparatus according to claim 1, wherein
the heating part is for heating the part to be heated by a length of one-time use or longer.

14. The sanitization apparatus according to claim 1, comprising
a use detecting sensor for detecting a use of the sanitization apparatus.

15. A doorknob, comprising the sanitization apparatus according to claim 1.

16. The sanitization apparatus according to claim 2, wherein
the part to be heated comprises a thin film.

17. The sanitization apparatus according to claim 2, wherein
the part to be heated is a polyimide film.

18. The sanitization apparatus according to claim 3, wherein
the part to be heated is a polyimide film.

19. The sanitization apparatus according to claim 4, wherein
the part to be heated is a polyimide film.

20. The sanitization apparatus according to claim 2, wherein
a thickness of the part to be heated is 30 μm or less.
